(19) United States
(12) Patent Application Publication
(10) Pub. No.: US 2025/0109018 A1
(43) Pub. Date: Apr. 3, 2025

(54) PRODUCTION OF CHEMICALS BY DIRECT PLASMA CONVERSION OF LIQUID FEEDSTOCKS

(71) Applicant: The Regents of the University of California, Oakland, CA (US)

(72) Inventors: Norleakvisoth Lim, Goleta, CA (US); Eric W. McFarland, Santa Barbara, CA (US); Michael J. Gordon, Goleta, CA (US)

(73) Assignee: The Regents of the University of California, Oakland, CA (US)

(21) Appl. No.: 18/904,421

(22) Filed: Oct. 2, 2024

Related U.S. Application Data
(60) Provisional application No. 63/587,290, filed on Oct. 2, 2023.

(51) Int. Cl. C01B 3/24; C01B 32/05; C07C 4/08

(52) U.S. Cl. CPC C01B 3/24; C01B 32/05; C07C 4/08

(57) ABSTRACT

A reaction system for carrying out a process for production of chemicals from liquid feedstocks includes a reactor containing one or more liquid reactants, an assembly with two or more electrodes within the reactor configured to generate a high voltage discharge within the one or more liquid reactants, an electrical power supply electrically coupled to the electrodes, inlets and outlets to the reactor for delivering reactants and removing products, and a pressure controller configured to control a pressure of the liquid reactants.

### CROSS-REFERENCE TO RELATED APPLICATIONS

The present application claims priority to (and the benefit under 35 U.S.C. Section 119(e) of) U.S. Provisional Patent Application No. 63/587,290 filed on Oct. 2, 2023 and entitled, “PRODUCTION OF CHEMICALS BY DIRECT PLASMA CONVERSION OF LIQUID FEEDSTOCKS,” the entire disclosure of which is incorporated herein by reference.

### STATEMENT REGARDING GOVERNMENTALLY SPONSORED RESEARCH OR DEVELOPMENT

None.

### BACKGROUND

A variety of important chemical transformations involving hydrocarbons and other feedstocks containing hydrogen make use of reactive intermediates including ions, radicals, and radical ions. A plasma is a non-equilibrium state of matter in which reactive species can be produced in a steady-state process and used for chemical transformations. Plasmas have been used previously for the processing of gases and liquids in a variety of chemical transformations; however, their use in the production of molecular hydrogen as well as other light hydrocarbons has been limited to gas phase processes at relatively low pressures.

### SUMMARY

In some embodiments, a reaction system for carrying out a process for production of chemicals from liquid feedstocks comprises a reactor containing one or more liquid reactants, an assembly with two or more electrodes within the reactor configured to generate a high voltage discharge within the one or more liquid reactants, an electrical power supply electrically coupled to the electrodes, inlets and outlets to the reactor for delivering reactants and removing products, and a pressure controller configured to control a pressure of the liquid reactants.

In some embodiments, a reactor process for producing chemicals from liquid feedstocks comprises generating a plasma between two or more electrodes within a liquid phase within a reactor vessel, circulating the liquid phase during the generating, generating reaction products in response to contacting the plasma with the liquid phase, and removing the reaction products from the reactor vessel. The liquid phase comprises one or more liquid phase reactants.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 illustrates a plasma discharge excited in liquid hydrocarbon.

FIG. 2 illustrates a plasma discharge and four phase region with electrodes configured as a tube through which the liquid hydrocarbon is pumped.

FIG. 3 illustrates how the tubular plasma electrode with liquid jet may be configured.

FIG. 4 schematically illustrates the plasma reaction vessel system configuration for continuous mode operation according to some embodiments.

FIG. 5 schematically illustrates the plasma reaction vessel system configuration for continuous mode operation for the production of H₂ and other gaseous olefin products according to some embodiments.

FIG. 6 illustrates how the plasma-liquid reaction vessel may be configured for operation with LNG reactant using liquid nitrogen cooling and additional heating to maintain the LNG at ˜111K.

FIG. 7 illustrates an example configuration of the plasma-liquid reaction system using multiple, tubular plasma excitation electrodes and liquid recirculation through the HV powered electrodes.

FIG. 8 illustrates an example configuration of the plasma-liquid reaction system using multiple, pin-like solid plasma excitation electrodes and liquid recirculation through a tubular common electrode.

FIG. 9 illustrates an example configuration of the plasma-liquid reaction system using multiple, pin-like solid plasma excitation electrodes and gas injection or sparging through the common electrode.

FIG. 10 illustrates an example configuration of the plasma-liquid reaction system where the liquid media where the discharge occurs may be a suspension, emulsion, or two-(liquid) phase.

FIG. 11 illustrates an example configuration of the plasma-liquid reaction system incorporating a photon capture device and a power conversion device.

FIG. 12 illustrates another example configuration of the plasma-liquid reaction system incorporating a photon capture device and a power conversion device.

FIG. 13 shows hexane aerosol formation under different liquid recirculation rates.

FIG. 14 is a photograph of the Example 1 experimental system.

FIG. 15 shows photographs and mass spectrometry data from Example 1.

FIG. 16 shows the data from analysis of the non-hexane liquid products analyzed via GC-MS from Example 1.

FIG. 17 shows an example of how changing the liquid jet flow speed, and hence the liquid-plasma contact time, can be used to modify the plasma characteristics and hence the reaction rate and/or product selectivities, in the Example 1 configuration using 20 kHz high voltage AC excitation.

FIG. 18 shows an example of how changing the high voltage AC excitation frequency (from 20 kHz to 19.2 kHz), and hence the reaction rate and/or product selectivities in the Example 1 configuration, can be used to modify the plasma characteristics, namely from a more low temperature plasma with blue emission from C₂ species at 20 kHz, to a more thermal plasma with intense blackbody emission from suspended carbon particulates at 19.2 kHz.

FIG. 19 shows an example of electrodes along with a generated plasma and corresponding gas phase during a plasma phase reaction.

FIG. 20 shows an exemplary mass spectrometry of components present with the plasma on an off during a reaction.

FIG. 21 shows an exemplary chart showing a gas product distribution during plasma conversion of hexane under different liquid recirculation rates and plasma voltages.

### DETAILED DESCRIPTION

The systems and methods disclose herein include processes and reactors for the chemical conversion of liquid-phase feedstocks into higher value chemicals using an electrically excited plasma generated within the liquid phase of the reactor. The disclosed systems and methods allow, for the first time, plasmas to be used inside of a liquid environment for generation of products including high-pressure gas phase products.

Disclosed herein are processes and reactors whereby liquids containing hydrogen are converted to molecular hydrogen and other products by the continuous electrical discharge and maintenance of a plasma within a liquid filled reactor. In contrast to previous uses of plasmas whereby plasma generated gas phase reactive intermediates are made to contact the surface of liquids, the disclosed systems and methods enable plasma processing within liquids maintained at high pressure to produce gas phase products at high pressure. The pressure of the liquid can be controlled to control the pressure and volume of the plasma. The liquid temperature can also be controlled to produce a range of pressures and temperatures, even where the plasma temperature is elevated relative to the surrounding liquid temperature.

Due to the high electronic temperatures possible in plasmas, reactions otherwise requiring temperatures far greater than commercially practical are possible. Water splitting reactions for example have been performed under modest conditions by injection of steam into a plasma torch. In some aspects, a part of the novelty disclosed herein is in the generation of a plasma in novel reactors within the liquid phase which may be maintained at high pressure to enable high pressure product gas generation.

Developing technologies to leverage the high energy density of liquid hydrocarbons, and especially the world's existing stockpile of natural gas, as well as its production and transportation infrastructure associated with liquefied natural gas (LNG), to produce clean H₂ and/or high value-added gaseous olefins—without the ancillary formation of CO₂—would be game changing.

The present application provides details that make use of a plasma discharge for chemical processing, applied in several electrical excitation configurations, which is carried out in a liquid feedstock. In some embodiments, a plasma generated within liquid hydrocarbon reactants creates a unique, multi-phase reaction environment, e.g., an environment where plasma (i.e., an ionized gas), gaseous H₂, gaseous and liquid hydrocarbons, and solid carbon are all present simultaneously, to produce clean H₂ and carbon. In some embodiments, the direct transformation of liquified natural gas (LNG) at its typical cryogenic storage conditions of 111K can be transformed into gaseous H₂ and solid, easy-to-separate carbon using electricity that can be provided from any source (e.g., the grid, green energy, etc.) and/or at points of use. The methodology may also be used to convert hydrocarbon liquids and their mixtures (e.g., C₆-C₁₈ alkanes and olefins) to hydrogen and solid carbon, and/or to high value gaseous olefins such as acetylene, ethylene, and their C₃-C₄ analogues. As such, direct conversion of electricity+LNG and/or liquid hydrocarbons to clean H₂ and solid carbon can be accomplished. Thus, the present disclosure is directly applicable to chemical process electrification and the use of clean and/or renewable energy (e.g., solar, wind, nuclear) to produce clean hydrogen fuel.

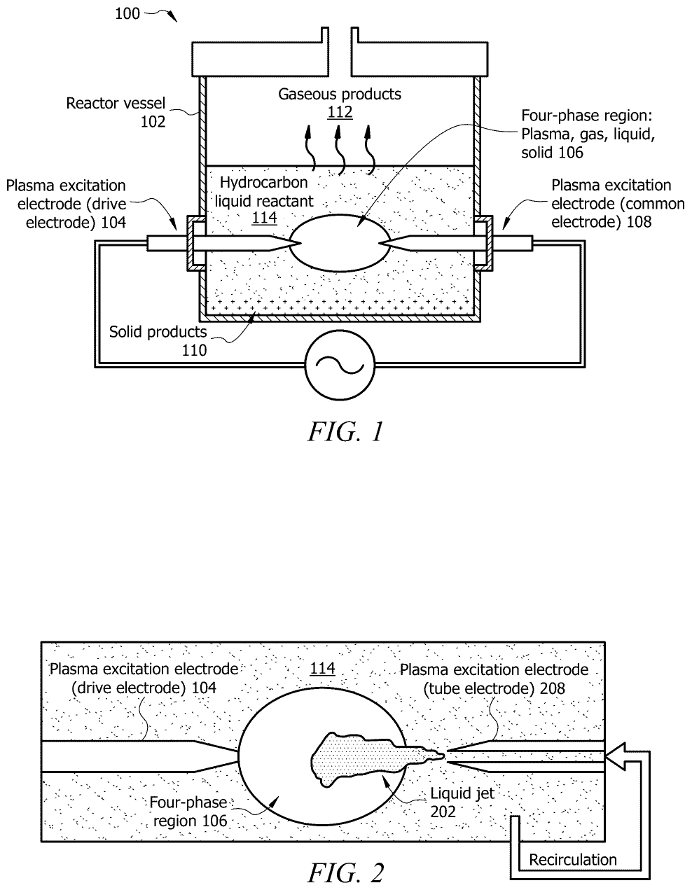

In an exemplary embodiment 100 as shown in FIG. 1, a plasma discharge 106 is excited in liquid hydrocarbon 114 between a high voltage (HV) AC “drive” electrode 104 and a “common” ground electrode 108, both electrically isolated, housed in a reactor vessel 102 that is appropriate for the temperatures and pressures needed to contain the liquid hydrocarbon 114 in a specific preferred state, e.g., at room temperature and atmospheric pressure for hexane or ˜111K and ˜4 psig for LNG. The circuit between the electrodes 104, 108 may be completed to earth directly or the common terminal of the excitation power supply, or both. Conversion of the hydrocarbon liquid 114 (reactant) to products occurs in a four phase region 106—comprising liquid reactants and products, gaseous products, solid products, and plasma—between and in the vicinity of the excitation electrodes 104, 108. Gaseous products 112 generated by plasma breakdown of the hydrocarbon 114, namely H₂ and/or short chain C₂-C₄ hydrocarbons, bubble up through the liquid and leave the reactor 102; solid products 110 remain suspended in the liquid reactant 114 and/or may settle out of the four phase region due to density differences.

The hydrocarbons used can comprise any hydrocarbons that can be in the liquid phase at the reaction conditions. As disclosed herein, the hydrocarbons can comprise any hydrocarbon liquids and their mixtures (e.g., C₆-C₁₈ alkanes and olefins) as well as any other hydrocarbon compounds comprising heteroatoms such as oxygen, nitrogen, sulfur, or the like (e.g., alcohols, amines, etc.). In some embodiments, additional reactants can comprise non-hydrocarbon molecules that contain the desired atomic makeup to provide for specific products. For example, oxygen can be introduced via the introduction of water with the hydrocarbons to produce oxygen containing hydrocarbons from the reaction such as alcohols, ethers, etc. The use of mixtures of hydrocarbons and other compounds can provide the reactants to produce both hydrogen and other reactants such as solid carbon and/or higher value chemicals.

The plasma-in-liquid described herein may be electrically initiated and sustained using various excitation schemes including, but not limited to, high voltage (HV) DC of different polarities; HV AC of various frequencies, ranging from about 60 Hz to greater than 100's kHz; radio frequencies (RF) of various frequencies (100s KHz to 100s MHz); microwaves; or pulsed DC or AC having various pulse widths, duty cycles, and frequencies; or any combination thereof. In the most general sense, the dielectric (liquid) gap between the plasma electrodes can be over-volted (electrically broken down) with high voltage to form an intermittent plasma discharge or arc that lowers the resistance between the electrodes, followed by sustained excitation of this plasma discharge using lower voltages in a continuous or pulsed fashion, depending on the electrical excitation scheme. In this context, “high voltage” refers to a voltage sufficient to form a plasma discharge or arc in the fluid between the electrodes.

The aforementioned plasma discharge may be initiated with various electrical circuit configurations including direct connection a DC or AC power source; connection to mechanical or electrical switching gear driven by various power supplies; via triggered or free-running spark gap, thyratron, or SCR; via a capacitive or inductive pulse forming network known by those trained in the art, or impedance matching network commonly encountered in RF plasmas and known by those trained in the art.

In some embodiments, one of the plasma excitation electrodes can be configured as a tube electrode 208, as shown in FIG. 2, through which the liquid hydrocarbon 114 from the vessel is recirculated so as to remove, via fluid flow, any solid carbon that may be deposited on or between the electrodes 106, 208, the latter being advantageous as a conductive carbon bridge between the electrodes 106, 208 may extinguish the plasma. For example, the solid carbon that forms during the reaction may form as a filament coupled to one of the electrodes 106, 208. In the event that the carbon filament bridges between the two electrodes 106, 208, a conductive pathway can be established to short the electrodes and extinguish the plasma. The liquid jet 202 exiting the tube electrode 208 can dislodge (e.g., clean) any solid carbon that may build up on or between the excitation electrodes 106, 208. In some aspects, the liquid jet 202 can be a high-speed jet of sufficient velocity to dislodge any solid carbon filaments that form during the reaction.

While shown in FIG. 2 as having the common electrode formed as a tube electrode, the drive electrode 104 can also be formed as a tube electrode. In some aspects, one or both of the electrodes can be formed as tube electrodes to allow for the injection or the liquid hydrocarbon 114 and/or other reactant streams, and any combination of one or more tube electrodes and reactant streams can be used in the embodiments disclosed herein.

In some aspects, one or more portions of the reactor system can be mechanically moved to help prevent the formation or bridging of solid products between the electrodes. For example, one or more of the electrodes and/or the reactor vessel itself can rotate, vibrate, or be mechanically agitated to prevent the buildup of solid carbon on the electrodes. In some aspects, an internal stirring mechanism or pump can be disposed within the reactor vessel. This may be in addition to or in place of the external fluid circulation through a tube type electrode or nozzle placed within the reactor vessel.

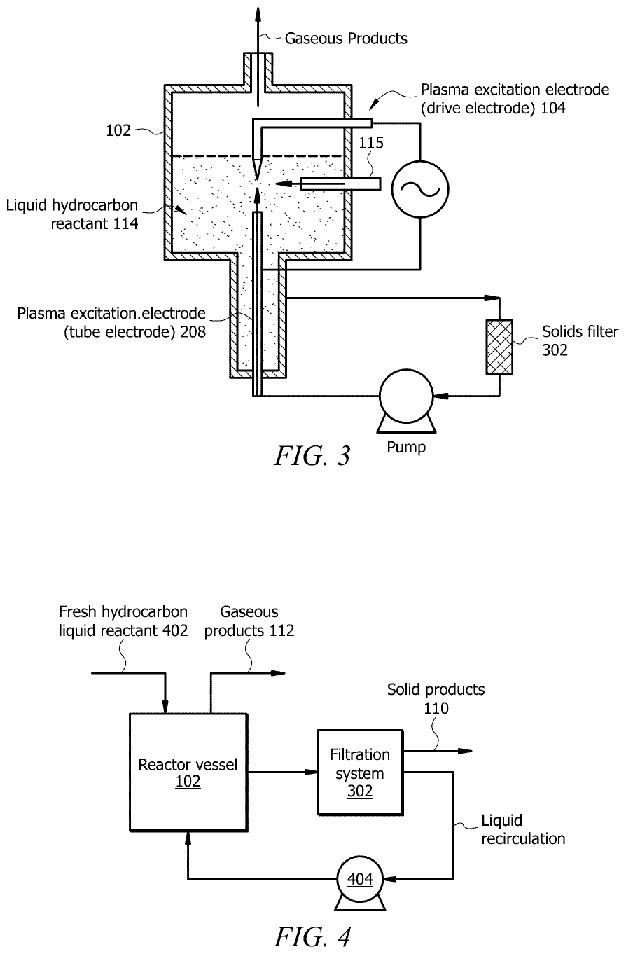

This liquid jet cleaning method can be implemented directly via the above as shown in FIG. 3, or implemented as an auxiliary tube 115 through which the hydrocarbon liquid reactant can be recirculated and directed toward the plasma excitation electrodes 106, 208 in various geometrical configurations. In another embodiment, a gas jet may be introduced through the liquid to prevent electrode deposition, and in another embodiment, inert solid particles suspended in the liquid jet may also be used to prevent electrode deposition. As shown in FIG. 3, a filter or other solids separation device 302 (e.g., settling chamber, etc.) can be used to remove the solid carbon in the hydrocarbon liquid 114 prior to recirculating the liquid 114 to the reactor vessel 102.

The liquid jet from the electrode 208 can also serve to mix the liquid hydrocarbon reactant 114 as well as minimize mass transfer resistances associated with delivery of reactants to the four phase reaction zone. Moreover, the geometrical configuration of the jet or jets used for reactant circulation may be specifically designed to control the contact time that the reactant has with the plasma and four phase region to tune the gaseous product distribution or selectivity. For example, the velocity of the jet, jet diameter, and orientation of the jet relative to the four phase region can be controlled to determine the time one or more reactants is within the four phase region. Further, a plurality of jets can be used to introduce different reactants to the four phase region, and the parameters of each jet can be designed to control the combined reactant composition within the four phase region.

The aforementioned plasma electrodes 104, 208 (and including electrode 108 as described herein) may take any geometric form (e.g., pins, tubes, concentric tubes, plates, mesh, or any combination thereof) and in any arrangement, e.g., as a linear or other array of many electrodes in different configurations, concentric rings, shell-and-tube configuration, or any combination thereof. These electrodes 104, 208 can be made of conductive and/or (refractory) materials such as, but not limited to, tungsten, molybdenum, stainless steel, steel, copper, aluminum, high temperature metal alloys, Elkonite (Cu—W composite), SiC, metal carbides (MoC₂, WC, etc.), conductive carbon (e.g., graphite, etc.), and/or metal nitrides (CrN, TiN, etc.).

As an alternate to the aforementioned embodiment utilizing conducting plasma electrodes 104, 208, other embodiments can be used where the plasma is excited in a capacitive fashion, similar to dielectric barrier plasma discharges (DBDs), where at least one of the plasma electrodes is electrically insulated via dielectric barrier from the plasma medium. In this case, the plasma would be excited using AC voltages in the few kHz to several 100 kHz range, or be applied as a repetitive, fast (sub microsecond) HV pulse.

FIG. 4 illustrates a process flow diagram of the overall process where the plasma reaction vessel 102 can be operated in continuous mode and solid products 110 (e.g., solid carbon, etc.) can be continually removed from the reactor vessel 102 via filtering of the recirculated liquid hydrocarbon (e.g., circulated using a pump 404, etc.) using a filtration system 302. In this embodiment, fresh hydrocarbon liquid 402 can be introduced as a feed stream that is continually added to the reaction vessel 102 to maintain the liquid level in the reaction vessel 102 as gaseous and solid products are generated and removed in stream 112.

In the various embodiments disclosed herein, the plasma reactor system may be operated at various temperature and pressure conditions, e.g., at a temperature from room temp to 600+° C., and/or at a pressure between about 1-50 bar, to facilitate enhancement of reaction rates, modify the produce gas distribution and or reaction selectivity, allow the use of alternative process feedstocks that are not necessarily in the liquid state at ambient conditions, and/or provide product gases to downstream processing at elevated pressure conditions to avoid additional compression equipment and costs. For example, the system may be operated in a manner to favor the formation of specific gaseous products (H₂ vs. C₂₊ olefins). As described herein, the selected reaction conditions can affect the volume and temperature of the four phase region, which can affect the product mix from the reactor system.

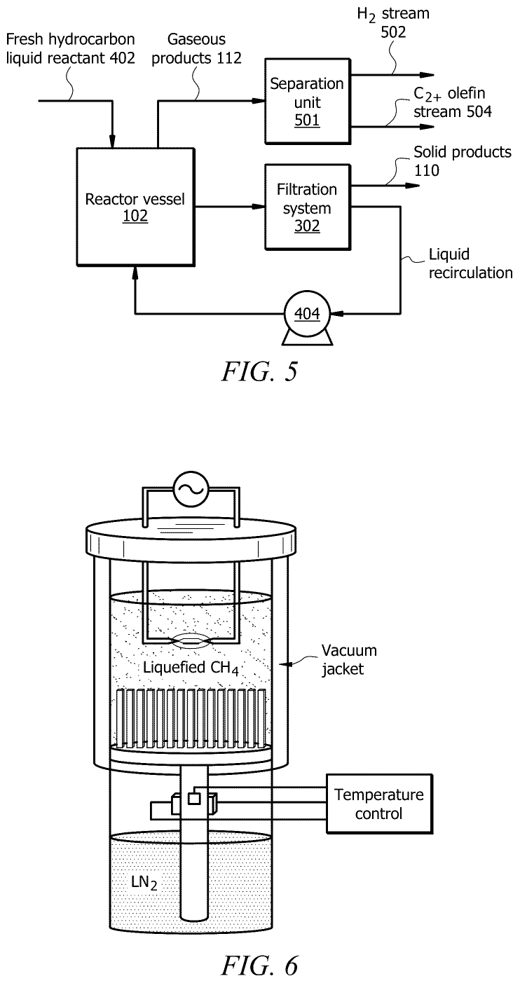

When products other than hydrogen and solid carbon are produced, a separation unit 501 can be used to produce a plurality of gas phase products. For example, these gaseous products could then be separated downstream of the plasma reactor, as detailed in FIG. 5. FIG. 5 is similar to the system of FIG. 4, and like components will not be re-described, but can be the same or similar to those described with respect to FIG. 4. As shown in FIG. 5, the separation unit 501 can receive the gaseous product stream 112 from the reactor 102 and separate the gaseous product stream 112 into one or more product streams. Any suitable separation units 501 can be used such as distillation, pressure or temperature swing adsorption, solvent extraction, or the like, which can occur in one or more physical separation units. Any plurality of streams can be produced depending on the product blend. As shown in FIG. 5, two streams comprising a hydrogen stream 502 and a C₂₊ olefin stream can be produced from the separation unit 501. When other products are produced in the reaction, different or additional streams can be separated and produced by the separation unit 501.

While described herein as operating in a continuous or semi-continuous mode, the plasma reaction vessel 102 can be operated in batch mode in some embodiments, where a fixed amount of liquid hydrocarbon can be converted to H₂ and solid carbon, or gaseous olefins and solid carbon. The reactor vessel 102 can then be opened to allow the solid and gaseous products to be removed before refilling the reactor vessel 102 for a subsequent batch conversion can take place.

In any of the aforementioned embodiments, the plasma reaction vessel temperature and pressure may be maintained at specific values to keep the hydrocarbon reactant in liquid form and/or to facilitate easy separation of the gaseous and solid products. When the hydrocarbon reactant is LNG specifically as shown in FIG. 6, the reaction vessel can be maintained at standard LNG cryogenic/transport conditions (˜111 K, 4 psig), which can facilitate the direct removal of H₂ as a gaseous product. As shown in FIG. 6, established methods of pumping and transporting LNG would be used to recirculate the liquid reactant as described above. This can include methods of keeping the LNG in a liquid state using a coolant with proper temperature controls. In some aspects, the reactor system can be installed at an LNG processing location (e.g., a liquefaction system and/or a gasification system) where LNG is readily available. When used with an LNG gasification system, the reactor system could be used to produce reaction products while also gasifying a portion of the LNG for use in a natural gas pipeline system.

The electrodes as described herein can be arranged in any suitable configurations to generate one or more four phase regions within the reactor vessel 102, where the configurations can be designed to provide a desired volume of the four phase regions. In some aspects, the plasma excitation electrodes may be configured as single electrodes or separated into multiple, geometrically separated excitation electrodes, the latter being individually connected to an external bank of individual capacitors in parallel that can be ultimately over-volted to initiate a discharge, in similar fashion to the excitation scheme used to create gas phase plasmas at atmospheric pressure in transversely excited atmospheric pressure (TEA) gas (CO₂) lasers.

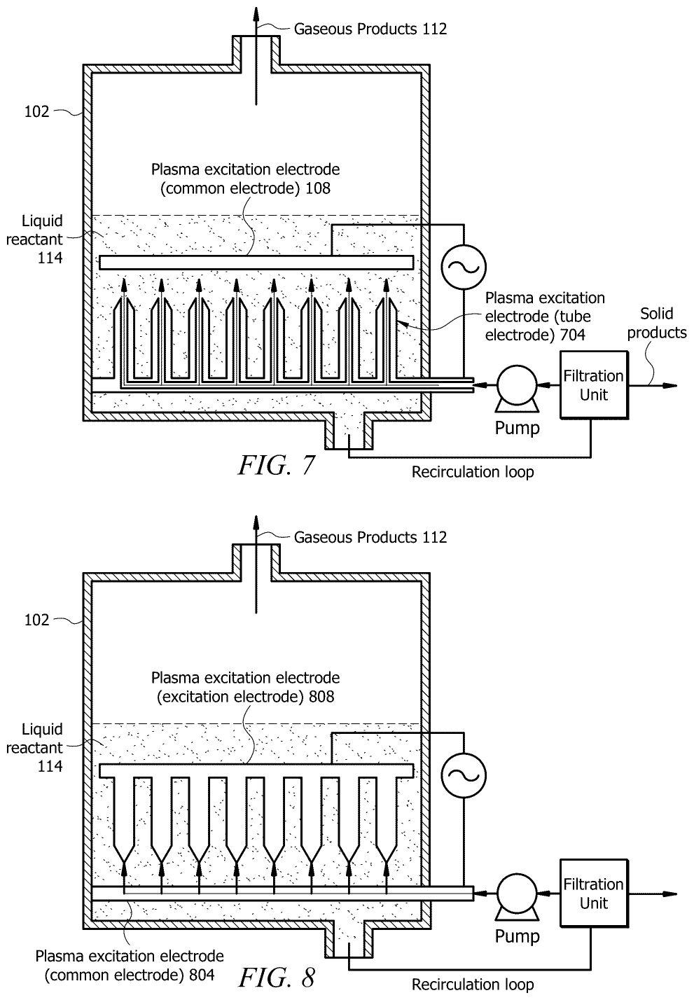

Exemplary configurations using one or more electrodes to produce a plurality of four phase regions are shown in FIGS. 7, 8, and 9. As shown in FIG. 7, one of the electrodes 704, 108 can be formed as a tube electrode comprising a plurality of points placed in proximity to the other electrode to allow for a plurality of four phase regions to be formed within the reactor vessel 102. The number of, the locations, and the spacing of the electrodes 704, 108 can be selected to provide a desired number of our phase regions in the reactor vessel 102. In some aspects, the electrodes can be separated by constant or time-varying gaps ranging from 0.1-10+ mm. While shown as having the drive electrode 704 forming the tube electrode, the common electrode 108 could alternatively or additionally be formed as a tube electrode in the embodiment shown in FIG. 7.

FIG. 8 illustrates a similar embodiment to that shown in FIG. 7, and like components will not be re-described in the interest of brevity. As shown in FIG. 8, the excitation electrode 808 can be formed with multiple points in proximity to a common electrode 804, which can be formed as a tube electrode. The resulting configuration can allow for a plurality of four phase regions to be formed within the reactor vessel 102.

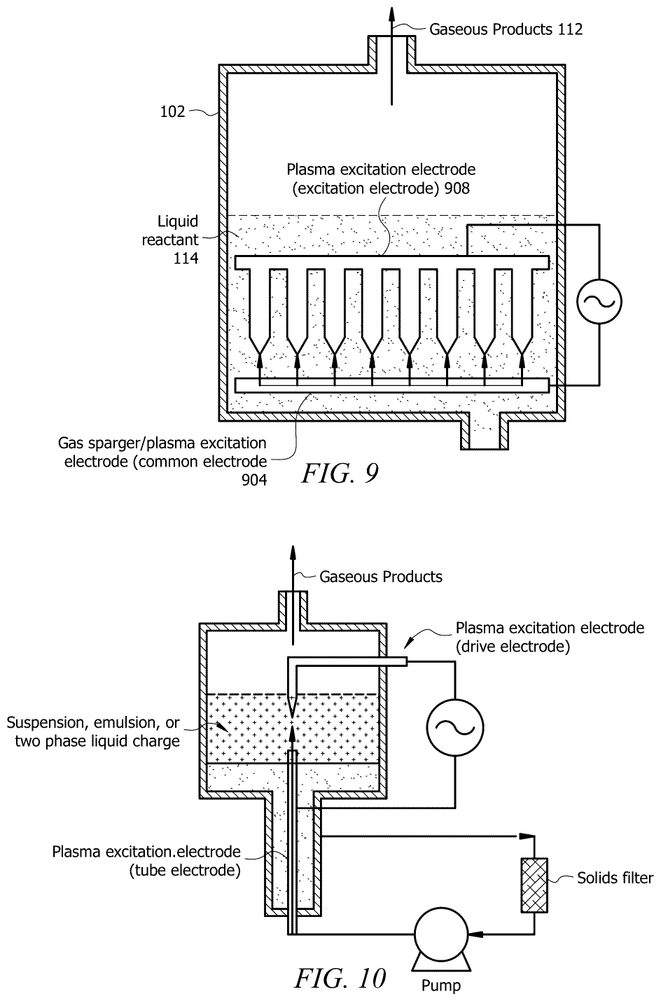

FIG. 9 illustrates a similar embodiment to that shown in FIGS. 7 and 8, and like components will not be re-described in the interest of brevity. As shown in FIG. 9, the excitation electrode 908 can be formed with multiple points in proximity to a common electrode 904, which can be formed as a tube electrode. The common electrode 904 can be formed as a gas sparger to allow a gas phase reactant to be introduced into the four phase region within the reactor vessel 102. When combined with the presence of the liquid phase reactant 114, various reaction products can be produced in the four phase regions. The resulting configuration can allow for a plurality of four phase regions to be formed within the reactor vessel 102, and the introduction of the gas phase reactants can be used to prevent the formation of carbon bridges from forming between the electrodes.

While FIGS. 7, 8, and 9 demonstrate the introduction or circulation of various fluids through one or both of the electrodes, the fluids (e.g., the recirculated liquid reactant 114) can be provided through one or more additional introduction points such as separate nozzles or jets arranged within the reactor vessel 102. In addition, each of FIGS. 7, 8, and 9 illustrate the use of a single common electrode and a single excitation electrode comprising a plurality of proximate locations to form four phase regions. In some aspects, a plurality of common electrodes and/or excitation electrodes can be used to form a plurality of four phase regions. The use of separate common and/or excitation electrodes can allow for four phase regions formed using different energy levels to be present within the reactor vessel 102.

In any of the aforementioned embodiments, gases, different fluids, fluids with entrained solid particles, and/or liquids with entrained solid particles may be c0-injected into the reactor vessel liquid and/or directly into the active four phase reaction environment between the plasma excitation electrodes, or via tubular plasma excitation electrodes (as outlined above). In this way, the co-injected gas, liquid and/or particles may be broken down in the plasma or liquid media and/or participate in reactions with the liquid media to make other specific products, not just limited to H₂ and other light hydrocarbons. The gas or liquid entrainment of solid particles into the active plasma zone (four phase region) may facilitate breakdown and/or conversion of the co-injected solids to smaller particles, compounds, or specific molecules of interest.

FIG. 10 illustrates an embodiment using a two phase fluid system as the reactant mixture within the reactor vessel. This embodiment may also involve the use of liquid reaction media that is a suspension, emulsion, or other two-phase liquid media. The use of a two-phase fluid (e.g., liquid-liquid, liquid-gas, etc.) can allow a specific mix of reactants to be present in the plasma to form desired products.

In some embodiments, the plasma may be generated within liquids other than hydrocarbons. For example, the plasma can be generated within liquid water to produce hydrogen and oxygen. At room temperature oxygen has 25× greater solubility in water than hydrogen and increases to more than 35× just above freezing. Oxygen produced in the plasma will be rapidly dissolved in the liquid water surrounding the plasma allowing for separation and prevention of recombination.

In another embodiment, the plasma can be contained in the liquid phase of molten salts or glasses including chlorides, sulfides, carbonates, nitrates, borates, hydroxides, their hydrates and their mixtures.

In another embodiment, the plasma can be contained in the liquid phase of an ionic liquid or mixtures of ionic liquids with other liquid phase reactants or media. This embodiment may be used to breakdown or assist in recycling and purifying ionic liquids or mixtures thereof, or to remove toxic compounds from waste streams.

In an alternative embodiment related to hydrogen carrier applications, the plasma conversion method may be applied to breakdown of liquid NH₃ to generate N₂ and H₂ fuel. Any of the configurations described herein can be used with a reactant or liquid comprising ammonia or similar compounds (e.g., urea, etc.). An extension of this embodiment may be applied to other “liquid” hydrogen carriers of current and future interest, such as hydrides, methylcyclohexane, or other partially or totally saturated hydrocarbon-based or other hydrogen carriers.

In an alternative embodiment related to water cleanup and/or waste remediation, the plasma in liquid water media or other aqueous mixtures will be used to synthesize H₂O₂, various peroxides, or other peroxide-like species that may be used as products directly, or to assist in chemical breakdown/modification of other (toxic) compounds present in or injected into the reactor vessel liquid media.

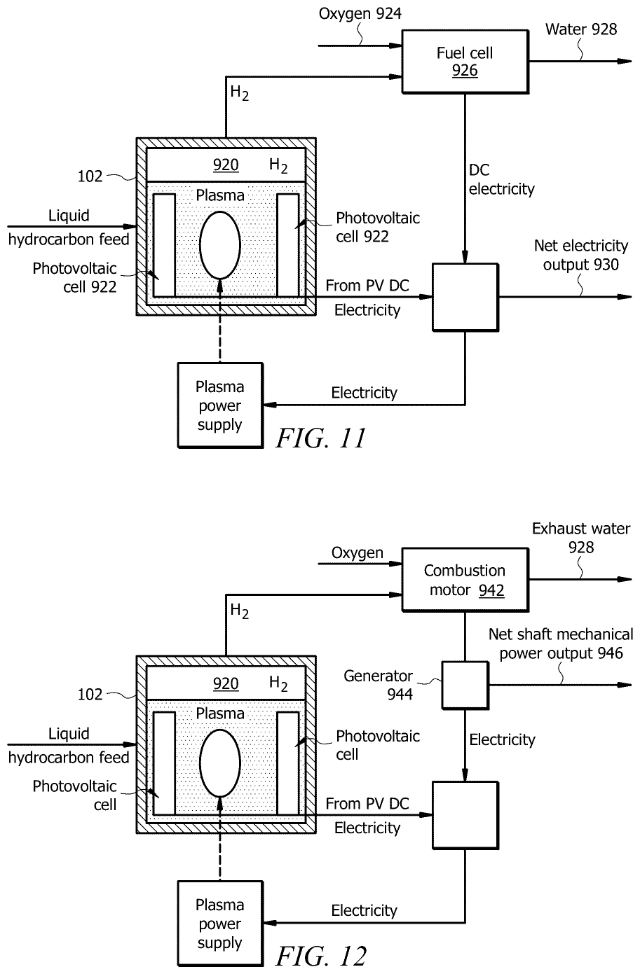

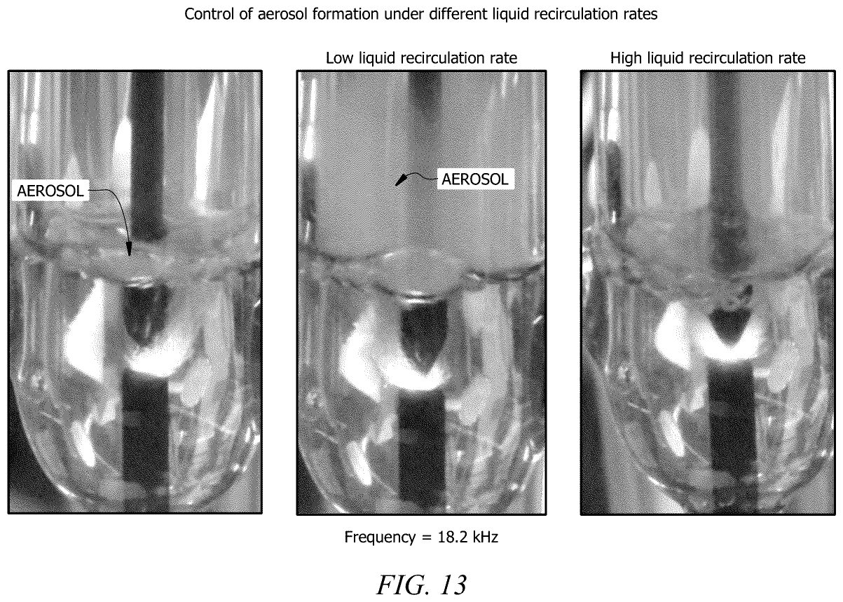

Electrical discharge plasmas produce photon energy (light) from a fraction of the energy input, and in most applications, this energy is lost. In some embodiments as schematically shown in FIG. 11, the relatively low temperature liquid environment surrounding the plasma 920 enables a photon conversion device 922 to be used. Suitable photon conversion devices 922 such as photovoltaic cells can be placed within the system (e.g., around the plasma within the reactor 102) to capture photons leaving the plasma zone and convert the photon energy to electricity. The photon capture could be used with any existing plasma technology.

In other embodiments, unique applications and configurations of the plasma pyrolysis process are used in systems to produce hydrogen for electricity or mechanic power generation. As shown in FIG. 11, the plasma zone 920 within a hydrocarbon liquid filled reactor 102 (e.g., filled with LNG, etc.), is surrounded by photo conversion devices 922 such as photovoltaic cells that can capture the light generated by the plasma 920 and convert the light to DC electricity. The hydrogen produced can be reacted with oxygen in stream 924, typically from air, in a fuel cell 926 to produce electricity and steam or liquid water in stream 928 in the fuel cell 926. The electricity from the fuel cell and the photovoltaic cells can be combined and a fraction of the electrical power used to generate the plasma while the excess electricity is exported as a product (e.g., electrical output 930). In one application, the system could be contained within a vehicle and the electricity used to power the vehicle. The details of the plasma and circulation of the liquid and removal of the solid carbon can include any of the embodiments described herein.

Another similar embodiment is shown schematically in FIG. 12 where instead of net electricity generation, motive power is produced from a combustion engine 942. Hydrogen from the plasma pyrolysis process in the reactor 102 (and possibly trace light hydrocarbons) can be delivered to a combustion engine 942 to generate an exhaust stream 928 and to power a mechanical process. A fraction of the mechanical power generated may be used to power a generator 944, where the generator's electrical output can be combined with the photovoltaic electrical output 946 to provide the input energy for the plasma.

Plasma systems can be used in the formation of plasmas in liquids, namely water, for underwater welding of steels, which involves the use of high current DC (200-500 A) and flux coated consumable welding electrodes. In this process, a plasma arc is struck between the welding electrode and work piece via direct contact to form an arc that partially melts the work piece and consumable welding electrode, resulting in the vaporization of water to form bubbles and an insulating gas/plasma envelope around the weld location. The present systems and methods are distinct from a welding system in several respects: (1) the plasma electrodes never come into contact, they are not consumed in the process, and the plasma reaction vessel does not function as a ‘work piece’; (2) HV AC is used to initiate and sustain the plasma, rather than direct contact of the electrodes and with the work piece; (3) high current DC at low voltage is not used; (4) the surrounding liquid medium functions as a liquid reactant, rather than a passive and unreactive liquid environment not involved in the process.

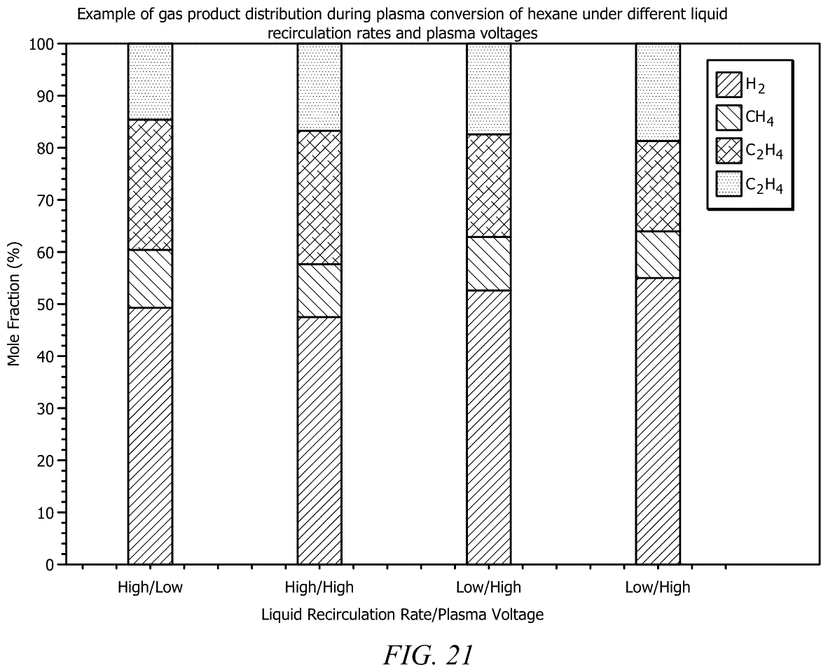

FIG. 21 shows an example of how hexane liquid reactant aerosols are formed and how they can be controlled by modifying the liquid recirculation rate. As indicated, liquid reactant aerosols can be formed inside of gas bubbles (vapor reactant and reaction products); these bubbles rise to the liquid surface and are ejected into the gas phase, potentially leading to mass transport of the liquid reactant out of the plasma reactor. A substantial quantity of aerosol is formed at low liquid recirculation rates; whereas, at high liquid recirculation rates, the formation of aerosols is reduced. Thus, the formation (and suppression) of aerosols, and therefore the quantity of liquid reactant leaving the plasma reactor, can be controlled by modifying the bubble residence time in the liquid phase via changing the recirculation rate and/or by controlling the liquid reactant height above the plasma phase region in the electrode gap; the latter allowing the bubble residence time to be varied appropriately for the desired effect or promoting or suppressing aerosol formation. The aforementioned aerosols may also be removed from the gas phase and/or gaseous product stream using standard techniques known by those trained in the art, e.g., via filtering, flow reversal, flow velocity changes, condensation, etc.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

### Plasma Within Liquid Hexane for Hydrogen Production

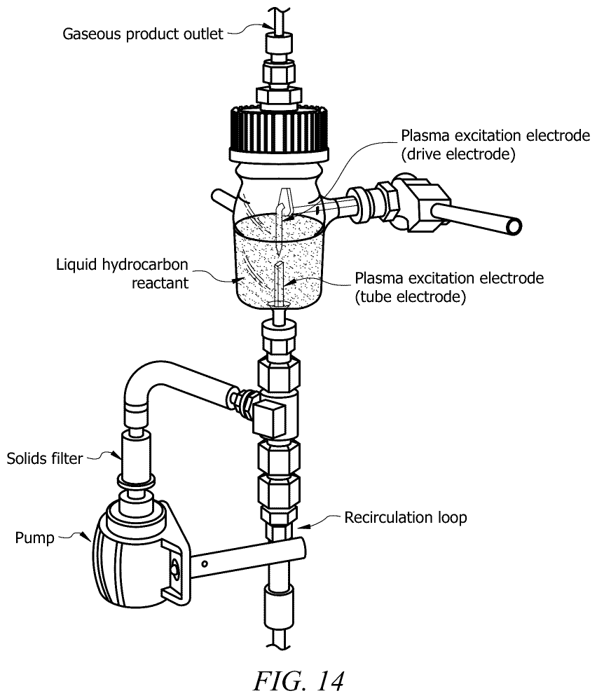

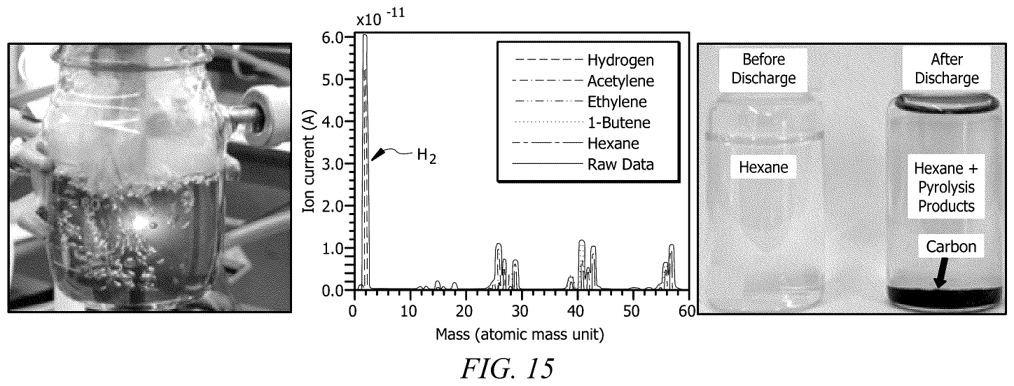

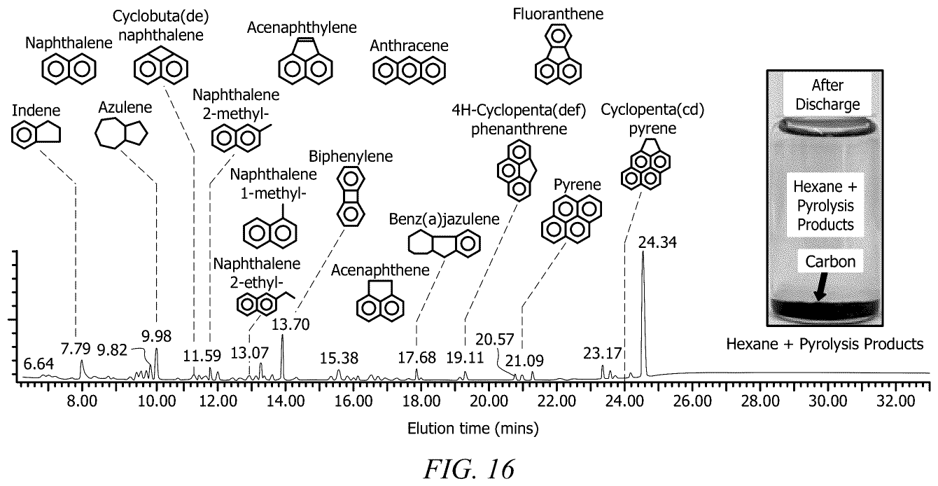

In a specific example, reference is made to FIGS. 3 and 14 that show a schematic and laboratory scale implementation of the recirculating liquid and tube electrode embodiment mentioned above, respectively. A proof-of-concept demonstration and data from the plasma reactor system in FIG. 14 is shown in FIG. 15a. For this experiment, the plasma was excited at 60 Hz AC frequency using an 18 kV step-up transformer in liquid hexane with ˜18 W of input power (estimated from the plasma current-voltage transient measured with HV oscilloscope probe and Regowski coil); ˜80-100 sccm of H₂ was generated, which is equivalent to an energetic input of ˜240 kJ/mol H₂=33 kW*hr/kg H₂. This energy requirement for an un-optimized system is considerably lower than for hydrogen production via water electrolysis, namely 40 kW*hr/kg H₂ (theoretical), but 60 kW*hr/kg H₂ in practice. Mass spectrometry analysis of the product gases (FIG. 15b) shows clear presence of a lot of H₂, with smaller levels of lighter olefins (acetylene, ethylene, 1-butene). FIG. 15c shows before and after photos of the reaction mixture; pure hexane (transparent, left) is converted to a yellow liquid (right) containing solid carbon that settles to the bottom of the container. The non-hexane liquid products in this yellow liquid were analyzed via GC-MS (FIG. 16), revealing that small levels of polycyclic aromatics are also formed by the plasma discharge in the liquid.

For the Example 1 case, ˜100 sccm of H₂ (as a lower bound) was generated from hexane with ˜18 W input power in a plasma volume of <1 mL, the latter estimated from videos of the plasma discharge region emitting light. This level of H₂ produced is equivalent to ˜74 mol H₂/m³ s (as a lower bound) in the aforementioned plasma volume. For a 100 kta H₂ plant, which is equivalent to a production rate of 1585 mol H₂/s, would therefore require a cumulative plasma reaction volume of 21.4 m³ (as an upper bound using the aforementioned 1 mL plasma volume as a reference).

### Example 2

### Modifying Plasma Operating Characteristics With Liquid Flow and Excitation Frequency

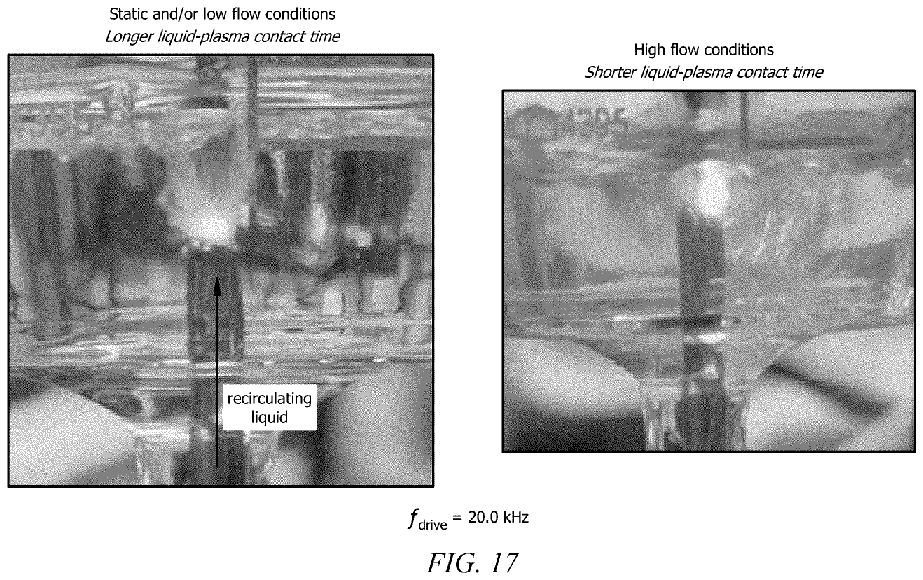

The ability to modify plasma operating characteristics and modes, and hence the reaction rates and/or product selectivities using different liquid recirculation rates, is shown in FIG. 17. In this case, the liquid jet exiting the tubular common electrode was very slow (or static) vs. very fast, and demonstrates a transition from a more blue color at low flow, associated with C₂ Swan band optical emission, to more white and red (H alpha emission at λ=656 nm) at high flows. In this way, the effective contact (or reaction) time the recirculating liquid spends in the four phase zone can be modified, thus providing the ability to tune reaction rates and/or products.

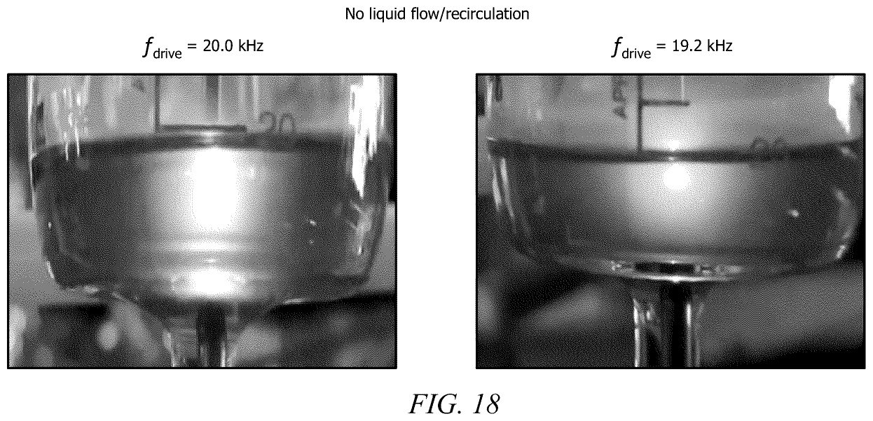

Plasma operating characteristics can also be affected in the static (no flow) case by changing the excitation frequency from 20 kHz to 19.2 kHz as show in FIG. 18. The plasma is more blue, associated with the aforementioned C₂ Swan band emissions, at 20 kHz; whereas, at 19.2 kHz excitation, the plasma region quickly becomes red-orange associated with a more thermal plasma with companion blackbody emission from suspended carbon particulates that are at high temperatures. In this way, the operating characteristics of the plasma may be modified, thus providing the ability to tune reaction rates and/or product distribution.

### Example 3

### Plasma Conversion of Hexanes

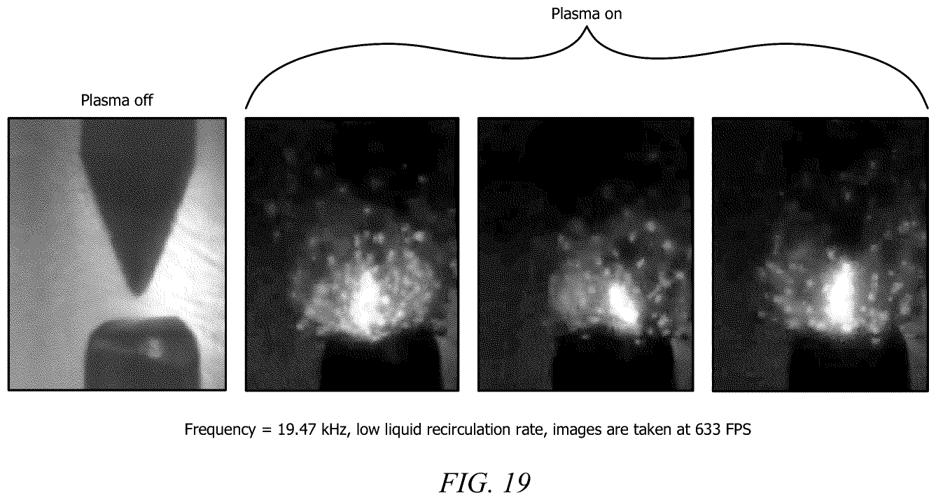

FIG. 19 shows an exemplary image of the electrodes used in the experimental setup with the current off along with the plasma discharge at an excitation frequency of 19.47 kHz with a low liquid recirculation rate. FIG. 19 also shows the generation of the plasma with images taken at 633 frames per second (FPS).

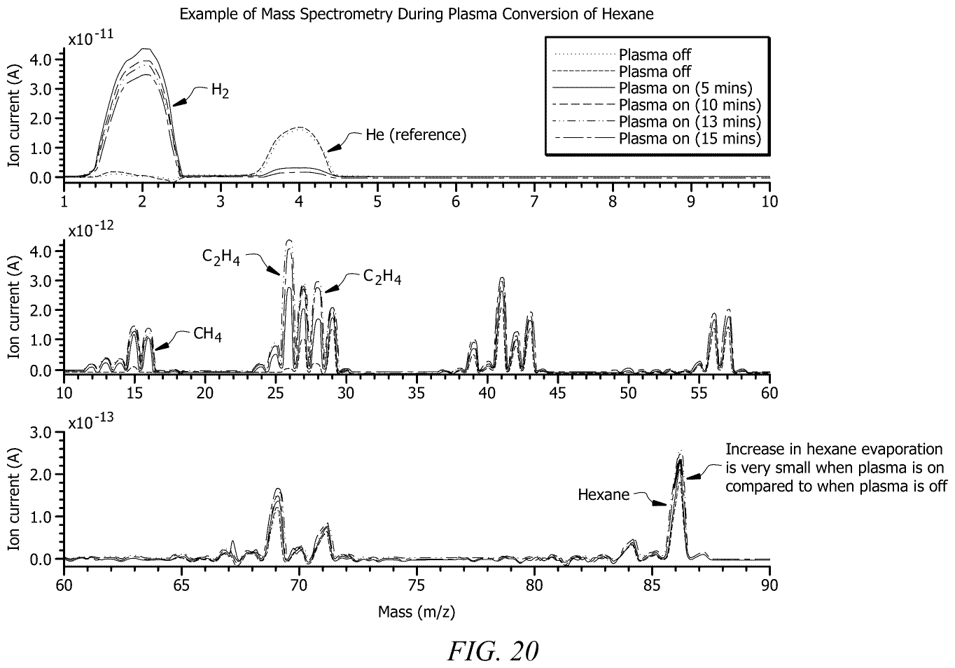

A set of exemplary mass spectroscopy traces for hexane conversion are shown in FIG. 20 as being overlaid. The plasma conversion was performed with the plasma off, the plasma on for 5 minutes, 10 minutes, 13 minutes, and 15 minutes. The results show the increased presence of hydrogen, methane, ethylene, and acetylene when the plasma is on relative to when the plasma is off. As also shown the increase in hexane evaporation in the traces when the plasma is on relative to when the plasma is off is relatively small. This shows that the plasma does not simply result in the evaporation of the hexane and rather produces reaction products.

FIG. 21 shows an exemplary gas product distribution resulting from the plasma conversion of hexane under different liquid recirculation rates and plasma voltages. As shown, a lower liquid recirculation rate tends to product a higher hydrogen output and a lower ethylene output. The plasma voltage does not have a great impact on the product distribution.

Having described various systems, reactors, and processes, certain aspect can include, but are not limited to:

In a first aspect, a reaction system for carrying out a process for production of chemicals from liquid feedstocks comprises: a reactor containing one or more liquid reactants; an assembly with two or more electrodes within the reactor configured to generate a high voltage discharge within the one or more liquid reactants; an electrical power supply electrically coupled to the electrodes; inlets and outlets to the reactor for delivering reactants and removing products; and a pressure controller configured to control a pressure of the liquid reactants.

A second aspect can include the reaction system of the first aspect, further comprising: a plasma formed by the high voltage discharge within the one or more liquid reactants.

A third aspect can include the reaction system of the first or second aspect, wherein the one or more liquid reactants are directly converted to the products within the plasma.

A fourth aspect can include the reaction system of any one of the first to third aspects, wherein the one or more liquid reactants comprise liquid hydrocarbons, their mixtures, or LNG, and wherein the one or more liquid reactants are directly converted to the products comprising gaseous products and solid carbon using the plasma.

A fifth aspect can include the reaction system of the fourth aspect, wherein the products comprise: gaseous hydrogen and solid carbon, or short chain, high value olefins such as acetylene, ethylene, or their C₃-C₄ analogs, or mixtures thereof and solid carbon, or high value liquid hydrocarbon products.

A sixth aspect can include the reaction system of any one of the first to fifth aspects, wherein an operating temperature and pressure are selected to maintain the liquid reactants comprising a hydrocarbon reactant in liquid form and/or to facilitate easy separation of products (gases and solids) from the liquid reactants comprising the hydrocarbon liquid reactant.

A seventh aspect can include the reaction system of any one of the first to fifth aspects, wherein an operating temperature and the pressure are selected to control the reaction rate of the reactants and/or gaseous product selectivity of the products.

An eighth aspect can include the reaction system of any one of the first to seventh aspects, wherein the two or more electrodes are separated by a gap, immersed in the liquid reactants comprising a hydrocarbon liquid reactant of interest, with the electrodes being driven electrically using a constant or variable high voltage (HV) and/or current, e.g., to form the discharge.

A ninth aspect can include the reaction system of any one of the first to eighth aspects, wherein the high voltage discharge is excited and sustained using different electrical drive configurations involving AC high voltage of fixed or variable frequency (10 Hz to 100's kHz) and/or amplitude, DC high voltage, or in a micro/nanosecond pulse configuration using various pulse frequencies, duty cycles, or pulse shapes and/or wherein the electrical power to the plasma may be delivered in a constant or variable voltage mode or in a constant or variable current mode.

A tenth aspect can include the reaction system of the ninth aspect, wherein the one or more electrodes are configured to be driven by multiple or different AC voltages whose frequency and/or phase are different and/or adjusted to facilitate higher reaction rate of the reactants, user specified product selectivity of the products, minimize carbon deposition, or higher energy efficiency of the overall process.

An eleventh aspect can include the reaction system of any one of the first to tenth aspects, wherein the high voltage discharge is operated using a selected electrical excitation scheme, temperature, and the pressure to form the products comprising a selected gaseous product (e.g., H₂ vs. C₂₊ olefins).

A twelfth aspect can include the reaction system of any one of the first to eleventh aspects, wherein the pressure controller is configured to control a pressure of a gas the one or more liquid reactants to control a gaseous product bubble rise time of the products comprising a gaseous product, a four phase region residence time, a product selectivity, or to facilitate downstream delivery, processing, and separation of the gaseous products.

A thirteenth aspect can include the reaction system of any one of the first to twelfth aspects, wherein the one or more liquid reactants comprise: liquefied natural gas or liquid methane, liquid alkanes, olefins, or aromatics, heavy liquid hydrocarbons, crude or refined oils, liquid petroleum distillates, gasoline or other petroleum-derived liquid fuels, or any mixtures thereof in any proportion, each at appropriate temperature and pressure conditions so they are in liquid form.

A fourteenth aspect can include the reaction system of any one of the first to thirteenth aspects, wherein the one or more liquid reactants in the reactor further comprise salts, catalysts, or conductive agents selected to modify a conductivity or a breakdown voltage of the one or more liquid reactants to facilitate initiating or sustaining the high voltage discharge, and/or to modify a rate of reaction of the reactants and product selectivity of the system.

A fifteenth aspect can include the reaction system of any one of the first to fourteenth aspects, further comprising: a gas phase disposed in the one or more liquid reactants, wherein the gas phase comprises one or more gases, both inert and/or reactive, selected to facilitate initiating or sustaining the discharge comprising a plasma discharge, and/or to modify the rate of reaction of the reactants and product selectivity of the process, or to synthesize the products comprising different gaseous products.

A sixteenth aspect can include the reaction system of any one of the first to fifteenth aspects, wherein the reactor has an elevated temperature and/or the pressure to permit the use of hydrocarbon sources that are solid or too viscous at ambient conditions to be used as the one or more liquid reactants.

A seventeenth aspect can include the reaction system of any one of the first to sixteenth aspects, wherein the two or more electrodes are made of a conductive material (e.g., graphite, stainless steel, refractory metals) and in the form of sharpened rods, needles, (sharpened) tubes, mesh, or plates of various geometry, or any combination thereof, or wherein the two or more electrodes are arranged in an array separated by constant or time-varying gaps ranging from 0.1-10+ mm.

An eighteenth aspect can include the reaction system of any one of the first to seventeenth aspects, wherein at least one electrode of the two or more electrodes is driven by HV, with the HV circuit completed through a separate common electrode or multiple common electrodes.

A nineteenth aspect can include the reaction system of any one of the first to eighteenth aspects, wherein at least one electrode of the two or more electrodes is in the form of a hollow or tube-like drive or common electrode, and where the liquid reactants comprising a hydrocarbon liquid in the reactor is configured to be circulated through the tube and recycled to the reactor.

A twentieth aspect can include the reaction system of any one of the seventeenth to nineteenth aspects, wherein the high voltage discharge is generated between two or more of the electrodes that are electrically isolated from the reactor vessel containing the liquid reactants.

A twenty first aspect can include the reaction system of any one of the seventeenth to nineteenth aspects, wherein the high voltage discharge is generated between the electrodes comprising one or more drive electrodes and a conducting reactor vessel, wherein the conducting reactor vessel forms a common electrode.

A twenty second aspect can include the reaction system of any one of the first to twenty first aspects, wherein one or more of the two or more electrodes are configured to rotate, vibrate, or be mechanically agitated to prevent the buildup of solid carbon on the electrodes.

A twenty third aspect can include the reaction system of any one of the first to twenty second aspects, wherein the reactor is configured to rotate, vibrate, or be mechanically agitated to prevent the buildup of solid carbon on a reactor surfaces.

A twenty fourth aspect can include the reaction system of any one of the first to twenty third aspects, further comprising: one or more photon capture devices, wherein the photon capture devices are disposed in view of the high voltage discharge.

A twenty fifth aspect can include the reaction system of the twenty fourth aspect, wherein the photon capture devices comprise a photovoltaic cell.

A twenty sixth aspect can include the reaction system of the twenty fourth or twenty fifth aspect, wherein the one or more photon capture devices are placed within the liquid reactants within the reactor, or directly outside of the reactor.

A twenty seventh aspect can include the reaction system of any one of the first to twenty sixth aspects, further comprising: hydrogen present in the reactor, wherein the high voltage discharge generates the hydrogen from the liquid reactants; and a conversion device configured to convert the hydrogen and generate electricity.

A twenty eighth aspect can include the reaction system of the twenty seventh aspect, wherein the conversion device comprises a fuel cell, wherein the fuel cell is configured to receive the hydrogen and an oxygen-containing stream and generate the electricity.

A twenty ninth aspect can include the reaction system of the twenty seventh aspect, wherein the conversion device comprises a generator.

A thirtieth aspect can include the reaction system of the twenty seventh aspect, wherein the conversion device comprises a combustion engine.

In a thirty first aspect, a reactor process for producing chemicals from liquid feedstocks comprises: generating a plasma between two or more electrodes within a liquid phase within a reactor vessel, wherein the liquid phase comprises one or more liquid phase reactants; circulating the liquid phase during the generating; generating reaction products in response to contacting the plasma with the liquid phase; and removing the reaction products from the reactor vessel.

A thirty second aspect can include the process of the thirty first aspect, wherein the reaction products comprise a gas phase product and a solid phase product.

A thirty third aspect can include the process of the thirty second aspect, further comprising: passing the liquid phase out of the reactor vessel; filtering the solid phase product out of the liquid phase; and returning the liquid phase to the reactor vessel.

A thirty fourth aspect can include the process of the thirty second or thirty third aspect, wherein the liquid phase comprises hydrocarbon reactants, wherein the solid phase comprises solid phase carbon, and wherein the gas phase product comprises hydrogen.

A thirty fifth aspect can include the process of any one of the thirty first to thirty fourth aspects, wherein at least a portion of the liquid phase is directly converted to the reaction products within the plasma.

A thirty sixth aspect can include the process of any one of the thirty first to thirty fifth aspects, wherein the process is operated in batch mode with a fixed charge of the liquid phase that is converted to the reaction products and solid carbon.

A thirty seventh aspect can include the process of any one of the thirty first to thirty sixth aspects, wherein the process is operated continuously with a continuous removal of solid products and a continuous addition of fresh liquid phase so as to maintain a constant or near constant volume of liquid hydrocarbon in the reactor vessel.

A thirty eighth aspect can include the process of any one of the thirty first to thirty sixth aspects, further comprising: stirring or circulating the liquid phase in the reactor vessel.

A thirty ninth aspect can include the process of any one of the thirty first to thirty eighth aspects, wherein the one or more liquid phase reactants comprise liquid hydrocarbons, their mixtures, or LNG, and wherein the one or more liquid reactants are directly converted to the reaction products comprising gaseous products and solid carbon using the plasma.

A fortieth aspect can include the process of any one of the thirty first to thirty ninth aspects, wherein the reaction products comprise: gaseous hydrogen and solid carbon, or short chain, high value olefins such as acetylene, ethylene, or their C₃-C₄ analogs, or mixtures thereof and solid carbon, or high value liquid hydrocarbon products.

A forty first aspect can include the process of any one of the thirty first to fortieth aspects, further comprising: controlling an operating temperature and pressure within the reactor vessel to maintain the liquid phase reactants comprising a hydrocarbon reactant in liquid form and/or to facilitate easy separation of reaction products (gases and solids) from the liquid phase reactants comprising a hydrocarbon liquid reactant.

A forty second aspect can include the process of any one of the thirty first to forty first aspects, wherein the two or more electrodes are separated by a gap, immersed in the liquid phase, with the electrodes being driven electrically using a constant or variable high voltage (HV) and/or current.

A forty third aspect can include the process of the forty second aspect, wherein the plasma is generated by a high voltage discharge that is excited and sustained using different electrical drive configurations involving AC high voltage of fixed or variable frequency (10 Hz to 100's kHz) and/or amplitude, DC high voltage, or in a micro/nanosecond pulse configuration using various pulse frequencies, duty cycles, or pulse shapes and wherein the electrical power to the plasma may be delivered in a constant or variable voltage mode or in a constant or variable current mode.

A forty fourth aspect can include the process of any one of the thirty first to forty third aspects, further comprising: driving the two or more electrodes by multiple or different AC voltages whose frequency and/or phase are different and/or adjusted to facilitate higher reaction rate of the liquid phase reactants, user specified product selectivity of the reaction products, minimize carbon deposition, or higher energy efficiency of the overall process.

A forty fifth aspect can include the process of any one of the thirty first to forty fourth aspects, wherein the reaction products comprise gaseous products and the process further comprising: controlling a pressure of a gas above the liquid phase in the reactor vessel to control a gaseous product bubble rise time of the gaseous products, a plasma residence time of the plasma, a product selectivity of the reaction products, or to facilitate downstream delivery, processing, and separation of the gaseous products.

A forty sixth aspect can include the process of any one of the thirty first to forty fifth aspects, wherein the one or more liquid reactants comprise: liquefied natural gas or liquid methane, liquid alkanes, olefins, or aromatics, heavy liquid hydrocarbons, crude or refined oils, liquid petroleum distillates, gasoline or other petroleum-derived liquid fuels, or any mixtures thereof in any proportion, each at appropriate temperature and pressure conditions so they are in liquid form.

A forty seventh aspect can include the process of any one of the thirty first to forty sixth aspects, wherein the one or more liquid phase reactants further comprise salts, catalysts, or conductive agents selected to modify a conductivity or a breakdown voltage of the one or more liquid reactants to facilitate initiating or sustaining the high voltage discharge, and/or to modify a rate of reaction and product selectivity of the system

A forty eighth aspect can include the process of any one of the thirty first to forty seventh aspects, wherein a gas phase is disposed in the liquid phase, wherein the gas phase comprises one or more gases, both inert and/or reactive, selected to facilitate initiating or sustaining the plasma comprising a plasma discharge, and/or to modify the rate of reaction of the products and product selectivity of the process, or to synthesize different gaseous products.

A forty ninth aspect can include the process of any one of the thirty first to forty eighth aspects, further comprising: liquifying a viscous or solid reactant to form the liquid phase.

A fiftieth aspect can include the process of any one of the thirty first to forty ninth aspects, further comprising: circulating the liquid phase through at least one electrode of the two or more electrodes.

A fifty first aspect can include the process of any one of the thirty first to fiftieth aspects, wherein the two or more electrodes are formed as an array to generate a plurality of plasmas within the reactor vessel.

A fifty second aspect can include the process of any one of the thirty first to fifty first aspects, further comprising: one or more photon capture devices, wherein the photon capture devices are disposed in view of the high voltage discharge.

A fifty third aspect can include the process of the fifty second aspect, wherein the photon capture devices comprise a photovoltaic cell.

A fifty fourth aspect can include the process of the fifty second or fifty third aspect, wherein the one or more photon capture devices are placed within the liquid reactants within the reactor.

A fifty fifth aspect can include the process of any one of the thirty first to fifty fourth aspects, wherein the reaction products comprise hydrogen, and wherein the process further comprises: converting the hydrogen to generate electricity.

A fifty sixth aspect can include the process of the fifty fifth aspect, wherein converting the hydrogen comprises: using a fuel cell, with an oxygen-containing stream; and generating the electricity.

A fifty seventh aspect can include the process of the fifty fifth aspect, wherein converting the hydrogen comprises: using a generator.

A fifty eighth aspect can include the process of the fifty fifth aspect, wherein converting the hydrogen comprises: using a combustion engine.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

What is claimed is:

1. A reaction system for carrying out a process for production of chemicals from liquid feedstocks comprising:
a reactor containing one or more liquid reactants;
an assembly with two or more electrodes within the reactor configured to generate a high voltage discharge within the one or more liquid reactants;
an electrical power supply electrically coupled to the electrodes;
inlets and outlets to the reactor for delivering reactants and removing products; and
a pressure controller configured to control a pressure of the liquid reactants.

2. The reaction system of claim 1, further comprising:
a plasma formed by the high voltage discharge within the one or more liquid reactants.

3. The reaction system of claim 1, wherein the one or more liquid reactants comprise liquid hydrocarbons, their mixtures, and/or liquified natural gas (LNG).

4. The reaction system of claim 3, wherein the products comprise:
gaseous hydrogen and solid carbon, or C₂-C₄ olefins and solid carbon, or liquid hydrocarbon products.

5. The reaction system of claim 1, wherein the two or more electrodes are separated by a gap, immersed in the liquid reactants comprising a hydrocarbon liquid reactant of interest, with the electrodes being driven electrically using a constant or variable high voltage (HV) and/or current to form the discharge.

6. The reaction system of claim 1, wherein the pressure controller is configured to control a pressure of a gas the one or more liquid reactants to control a gaseous product bubble rise time of the products comprising a gaseous product, a four phase region residence time, a product selectivity, and/or to facilitate downstream delivery, processing, and/or separation of the gaseous products.

7. The reaction system of claim 1, wherein the one or more liquid reactants in the reactor further comprise at least one of salts, catalysts, or conductive agents selected to modify a conductivity or a breakdown voltage of the one or more liquid reactants to facilitate initiating or sustaining the high voltage discharge, and/or to modify a rate of reaction of the reactants and product selectivity of the system.

8. The reaction system of claim 1, further comprising:
a gas phase disposed in the one or more liquid reactants, wherein the gas phase comprises one or more gases, both inert and/or reactive, selected to facilitate initiating or sustaining the discharge comprising a plasma discharge, and/or to modify the rate of reaction of the reactants and product selectivity of the process, or to synthesize the products comprising different gaseous products.

9. The reaction system of claim 1, wherein at least one electrode of the two or more electrodes is in the form of a hollow or tube-like drive or common electrode, and where the liquid reactants comprising a hydrocarbon liquid in the reactor is configured to be circulated through the tube and recycled to the reactor.

10. The reaction system of claim 1, further comprising:
one or more photon capture devices, wherein the photon capture devices are disposed in view of the high voltage discharge, wherein the photon capture devices comprise a photovoltaic cell, and wherein the one or more photon capture devices are placed within the liquid reactants within the reactor, or directly outside of the reactor.

11. A reactor process for producing chemicals from liquid feedstocks, the process comprising:
generating a plasma between two or more electrodes within a liquid phase within a reactor vessel, wherein the liquid phase comprises one or more liquid phase reactants;
circulating the liquid phase during the generating;
generating reaction products in response to contacting the plasma with the liquid phase; and
removing the reaction products from the reactor vessel.

12. The process of claim 11, wherein the reaction products comprise a gas phase product and a solid phase product, and wherein the process further comprises:
passing the liquid phase out of the reactor vessel;
filtering the solid phase product out of the liquid phase; and
returning the liquid phase to the reactor vessel.

13. The process of claim 11, wherein at least a portion of the liquid phase is directly converted to the reaction products within the plasma.

14. The process of claim 11, wherein the process is operated in batch mode with a fixed charge of the liquid phase that is converted to the reaction products and solid carbon.

15. The process of claim 11, wherein the process is operated continuously with a continuous removal of solid products and a continuous addition of fresh liquid phase so as to maintain a constant or near constant volume of liquid hydrocarbon in the reactor vessel.

16. The process of claim 11, further comprising:
stirring or circulating the liquid phase in the reactor vessel.

17. The process of claim 11, wherein the reaction products comprise:
gaseous hydrogen and solid carbon, or C₂-C₄ olefins and solid carbon, or liquid hydrocarbon products.

18. The process of claim 11, further comprising:
driving the two or more electrodes by multiple or different AC voltages whose frequency and/or phase are different and/or adjusted to facilitate higher reaction rate of the liquid phase reactants, user specified product selectivity of the reaction products, minimize carbon deposition, or higher energy efficiency of the overall process.

19. The process of claim 11, wherein the reaction products comprise gaseous products and the process further comprising:
controlling a pressure of a gas above the liquid phase in the reactor vessel to control a gaseous product bubble rise time of the gaseous products, a plasma residence time of the plasma, a product selectivity of the reaction products, and/or to facilitate downstream delivery, processing, and separation of the gaseous products.

20. The process of claim 11, further comprising:
one or more photon capture devices, wherein the photon capture devices are disposed in view of the high voltage discharge.
